(19) United States
(12) Patent Application Publication
(10) Pub. No.: US 2022/0347168 A1
(43) Pub. Date: Nov. 3, 2022

(54) LUNG TARGETED ANTICANCER THERAPIES WITH LIPOSOMAL ANNAMYCIN

(71) Applicant: Board of Regents, The University of Texas System, Austin, TX (US)

(72) Inventors: Waldemar PRIEBE, Houston, TX (US); Rafal ZIELINSKI, Spring, TX (US); Izabela FOKT, Houston, TX (US); Stanislaw SKORA, Spring, TX (US)

(21) Appl. No.: 17/663,133

(22) Filed: May 12, 2022

Related U.S. Application Data
(63) Continuation of application No. PCT/US2020/061775, filed on Nov. 23, 2020.
(60) Provisional application No. 62/938,845, filed on Nov. 21, 2019.

(51) Int. Cl. A61K 31/475; A61K 38/17; A61P 35/00; A61K 31/704; A61K 33/24; A61K 31/675; A61K 9/127

(52) U.S. Cl. CPC A61K 31/475; A61K 38/1719; A61P 35/00; A61K 9/127; A61K 33/24; A61K 31/675; A61K 31/704

(57) ABSTRACT

Provided is a method of treating cancer cells localized in the lung by administering to such patients a therapeutically effective amount of a liposomal annamycin formulation (L-Ann).

This application is a bypass continuation of International Application No. PCT/US2020/061775, filed Nov. 23, 2020, which claims priority to, and the benefit of, U.S. Application No. 62/938,845, filed Nov. 21, 2019, the entireties of each are incorporated by reference herein.

Annamycin is a non-cardiotoxic anthracycline antibiotic with unique biological properties. Our earlier studies showed that Annamycin is not cross-resistant with doxorubicin (DOX) and is a poor substrate for P-glycoprotein 1 (P-gp) [aka ATP-binding cassette sub-family B member 1 (ABCB1) or multidrug resistance protein 1 (MDR1)], a major mechanism of DOX resistance in different types of cancer. Annamycin, in contrast to DOX, achieves relatively high levels of cellular accumulation, especially in multidrug resistant (MDR) cell lines, and induces significant DNA damage in cancer cells including MDR cells. Clinically, Annamycin is administered in a liposomal formulation (L-Annamycin) the in vivo activity of which has been demonstrated in different tumor models.

Surprisingly, pharmacokinetic and organ distribution studies of Annamycin and L-Annamycin formulated in liposomes (L-Annamycin) revealed unexpectedly high levels of Annamycin in lung tissue. Annamycin levels (AUC, 24 h) in lungs were over 10-fold greater than in plasma. Importantly, high lung uptake Annamycin resulted in levels exceeding 6 to 7-fold those of DOX. Annamycin's high intracellular uptake and unique distribution might in part be responsible for high L-Annamycin's activity against MDR cancer cells.

Accordingly, provided is a method of treating cancer in the lung comprising administering to a patient in need thereof a therapeutically effective amount of liposomal annamycin.

These and other aspects of the disclosure disclosed herein will be set forth in greater detail as the patent disclosure proceeds.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the Figures below, error bars represent standard error of the mean; arrows indicate administration points.

FIG. 1 shows PK and biodistribution analysis of Annamycin in lungs and plasma after single intravenous administrations of the drug. Error bars represent standard error of the mean (SEM).

FIGS. 2-7 show the tumor progression and survival of CT26 tumor-bearing mice treated with L-annamycin. FIGS. 2-4 and 5 each represent a longitudinal analysis of BLI signal for mice treated with L-Ann at two different dosing levels: 2 and 4 mg/kg. FIG. 6 shows the distribution of BLI on day 58 after tumor implantation. FIG. 7. shows Kaplan-Mayer analysis (percent survival) of CT26 tumor bearing mice treated with L-Annamycin.

FIG. 8 shows the tumor progression and survival of 4T1 tumor-bearing mice treated with L-Annamycin.

FIG. 9 shows a longitudinal analysis of BLI signal for 4T1 tumor bearing mice treated with L-Ann.

FIG. 10 shows Kaplan-Mayer analysis (percent survival) of 4TI tumor bearing mice treated with L-Annamycin.

### DETAILED DESCRIPTION

Disclosed herein are methods of treating cancer in the lung (lung-localized tumors) comprising administering to a patient in need thereof a therapeutically effective amount of liposomal annamycin.

In certain embodiments, treating cancer in the lung comprises one or more of the following: (a) increasing survival time of the patient; (b) reducing volume of the primary cancer; (c) retarding growth of the primary cancer; (d) reducing number of metastatic tumors; (e) reducing volume of metastatic tumors; and (f) retarding growth of metastatic tumors.

In certain embodiments, treating cancer in the lung does not result in annamycin-induced toxicity of the lung of such severity that repeated administration of the annamycin is contraindicated.

In certain embodiments, treating cancer in the lung does not result in annamycin-induced systemic toxicity of such severity that repeated administration of the annamycin is contraindicated.

In certain embodiments, the patient has primary or metastatic cancer in the lung.

In certain embodiments, the patient has primary cancer in the lung. In certain embodiments, the primary lung cancers is small-cell lung cancer (SCLC) or non-small-cell lung cancer (NSCLC). In certain embodiments, the non-small-cell lung cancer is chosen from adenocarcinoma, squamous cell carcinoma, large cell carcinoma, and undifferentiated NSCLC.

In certain embodiments, the patient has metastatic cancer in the lung. Metastatic lung cancers can originate in nearly any other tissue and spread to the lung. For example, the metastasized cancer may be a breast or colon cancer. In certain embodiments, the metastatic cancer is a metastasis of a primary cancer selected from bladder cancer, breast cancer, colorectal cancer, head and neck cancer, kidney cancer, melanoma, pancreatic cancer, prostate cancer, and ovarian cancer. In certain embodiments, the metastatic cancer is a metastasis of a sarcoma. In certain embodiments, the metastatic cancer is from a cancer of unknown primary origin.

In certain embodiments, the cancer in the lung is mesothelioma.

The methods involve administering to a mammal an effective amount of drug compositions. The administering step can suitably be parenteral and by intravenous, intraarterial, intramuscular, intralymphatic, intraperitoneal, subcutaneous, intrapleural, intrathecal injection, or by topical application dosage. In some embodiments, such administration is repeated regimen until tumor regression or disappearance is achieved, and may be used in conjunction with forms of tumor therapy such as surgery or chemotherapy with different agents.

In some embodiments, the dose administered is between approximately 125 and 280 mg/m² with respect the mammalian subject to which it is administered.

In certain embodiments, the administering is repeated weekly. In certain embodiments, the administering is repeated every two, three, or four weeks.

In certain embodiments, the method further comprises administering an effective amount of at least one chemotherapeutic agent to the subject.

In certain embodiments, the at least one chemotherapeutic agent is selected from actinomycin, afatinib, alectinib, asparaginase, azacitidine, azathioprine, bicalutamide, bleomycin, bortezomib, camptothecin, carboplatin, capecitabine, certinib, cetuximab, cisplatin, chlorambucil, crizotinib, cyclophosphamide, cytarabine, daunorubicin, docetaxel, doxifluridine, doxorubicin, erlotinib, epirubicin, epothilone, etoposide, fludarabine, flutamine, fluorouracil, fostamatinib, gefitinib, gemcitabine, hydroxyurea, ibrutinib, idarubicin, ifosfamide, imatinib, ipilimumab, irinotecan, lapatinib, letrozole, mechlorethamine, mercaptopurine, methotrexate, mitomycin, mitoxantrone, nilotinib, octreotide, oxaliplatin, paclitaxel, palbociclib, panitumumab, pemetrexed, raltitrexed, selumetinib, sorafenib, sunitinib, tamoxifen, temozolomide, teniposide, tioguanine, topotecan, trastuzumab, tremelimumab, valrubicin, vemurafenib, vinblastine, vincristine, vindesine, vinorelbine, and combinations thereof.

In certain embodiments, the at least one chemotherapeutic agent comprises a combination selected from:
cyclophosphamide, doxorubicin, and vincristine;
mitomycin, vindesine and cisplatin;
cisplatin and vinorelbine; and
cisplatin, etoposide and ifosfamide.

In certain embodiments, the method further comprises administering an effective amount of at least one immunotherapeutic agent to the subject. Examples of immunotherapy include, but are not limited to, monoclonal antibodies, immune checkpoints inhibitors, cancer vaccines and non-specific immunotherapy. In certain embodiments, the at least one immunotherapeutic agent is selected from chloroquine, hydroxychloroquine, Picibanil, Krestin, Schizophyllan, Lentinan, Ubenimex, an interferon, an interleukin, macrophage colony stimulating factor, granulocyte colony stimulating factor, erythropoietin, lymphotoxin, BCG vaccine, Corynebacterium parvum, Levamisole, Polysaccharide K, Procodazole, an anti-CTLA4 antibody (e.g., ipilimumab, tremelimumab), an anti-PD-1 antibody (e.g., nivolumab, pembrolizumab), and an anti-PD-L1 antibody.

In certain embodiments, the method further comprises one or both of resecting the lung cancer and administering radiation therapy.

Annamycin is provided in a liposomal-based formulation. In certain embodiments, the liposomal annamycin comprises annamycin, one or more lipids, and one or more non-ionic surfactants.

The lipid may comprise one or more phospholipids, which form micelles and lipid bilayers and are widely used to prepare liposomal, ethosomal and other nano-formulations. Examples of suitable phospholipids include dimyristoylphosphatidylcholine (DMPC) and dimyristoylphosphatidylglycerol (DMPG).

In certain embodiments, the lipids comprise Dimyristoylphosphatidylcholine (DMPC) and dimyristoylphosphatidylglycerol (DMPG), and the non-ionic surfactant comprises a polysorbate-type surfactant formed from the ethoxylation of sorbitan followed by the addition of a carboxylic acid.

Non-ionic surfactants typically have covalently bonded oxygen-containing hydrophilic groups, which are bonded to hydrophobic parent structures. The nonionic surfactant may comprise a polysorbate-type surfactant formed from the ethoxylation of sorbitan followed by the addition of a carboxylic acid, such as polyoxyethylene sorbitan monolaurate (e.g., polysorbate 20).

In certain embodiments, the non-ionic surfactant comprises polyoxyethylene sorbitan monolaurate.

In certain embodiments, the liposomal annamycin is provided as a preliposomal lyophilizate composition that is reconstituted into an aqueous liposome composition through hydration, as described, e.g., in U.S. Pat. No. 7,238,366 which is incorporated by reference in its entirety for all purposes.

In certain embodiments, the preliposomal annamycin lyophilizate comprises:
1.8-2.2 wt % Annamycin;
3.0-3.4 wt. % Polysorbate 20; and
94.4-95.2 wt. % of lipids selected from DMPC and DMPG.

In certain embodiments, the DMPC is 65.3-67.3 wt. % and the DMPG is 27.1-29.9 wt. %.

In certain embodiments, each of these amounts may vary somewhat, for example plus or minus 10% of the weight percent given.

### Definitions

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “Annamycin” shall mean the compound, (7S,9S)-7-(((2R,3R,4R,5R,6S)-4,5-dihydroxy-3-iodo-6-methyltetrahydro-2H-pyran-2-yl)oxy)-6,9,11-trihydroxy-9-(2-hydroxyacetyl)-7,8,9,10-tetrahydrotetracene-5,12-dione, having the following structure:

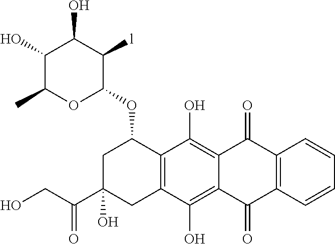

The term “high purity Annamycin preliposomal lyophilizate” shall mean purity of material which is no less than 95% Annamycin as analyzed by HPLC using a verified standard sample. In some embodiments, the Annamycin is at least 96% pure, or at least 97% pure, or at least 98% pure, or at least 99% pure.

The term “liposomes,” “liposomal,” and the like shall mean generally spherical structures comprising lipids, fatty acids, lipid bilayer type structures, unilamellar vesicles and amorphous lipid vesicles. Classically, liposomes are completely closed lipid bilayer membranes containing an entrapped aqueous volume. Liposomes include non-classical forms where the Annamycin may be inside the bilayer, part of the bilayer and absorbed onto the bilayer. Liposomes may be unilamellar vesicles (possessing a single bilayer membrane) or multilamellar vesicles (onion-like structures characterized by multiple membrane bilayers, each separated from the next by an aqueous layer). The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “head” orient towards the aqueous phase.

The term “preliposome-lyophilizate” and “preliposomal lyophilizate” shall mean a non-aqueous material that will form liposomes upon addition of aqueous solution. In some embodiments the non-aqueous material is dry (as in non-liquid, non-gel) material. Lyophilizate is used expansively to include the dry residue of sublimation of frozen liquids from non-volatile materials, the residue of roto evaporation and similar procedures, and dry compositions that, upon addition of an aqueous phase (with or without agitation) with result in liposomes. It is particularly to be understood that “preliposome-lyophilizate” is not in liposomal form after lyophilization.

The term “lipids” refers to any of a class of pharmaceutically acceptable organic compounds that are fatty acids or their derivatives. In some embodiments, the lipids are phospholipids, such as phosphatidylcholines including DMPC and DPMG, but may also include other lipids, such as egg phosphatidylethanolamine.

The term “non-ionic surfactants” refers to pharmaceutically acceptable surfactants that have covalently bonded oxygen-containing hydrophilic groups, which are bonded to hydrophobic parent structures. Suitable non-ionic surfactants include ethoxylates, fatty alcohol ethoxylates, alklphenol ethoxylates, fatty acid ethoxylates, ethoxylated fatty esters and oils, ethoxylated amines, fatty acid amides, terminally blocked ethoxylates, poloxamers, fatty acid esters of polyhydroxy compounds, fatty acid esters of glycerol, and fatty acid esters of sorbitol. In some embodiments, the non-ionic surfactants are polysorbate-type surfactants formed from the ethoxylation of sorbitan followed by the addition of a carboxylic acid. In some embodiments, the non-ionic surfactant comprises polyoxyethylene sorbitan monolaurate (Polysorbate 20), and polyethoxylated sorbitan monooleic acid (Polysorbate 80).

“Polysorbate 20” refers to a commercially available nonionic surfactant (ICI Americas Inc.) consisting of a mixture of different length chains of polyoxyethylene linked to a common sorbitan sugar. These polyoxyethylene sugars are also linked to a fatty acid. A tradename for this material is Tween™ 20; the composition is polyoxyethylene sorbitan monolaurate (MW approximately 1300). As Polysorbate 20 is shown below, w+x+y+z=20.

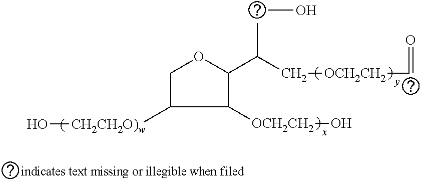

The term “pharmaceutically acceptable acid” refers to any organic and inorganic acid that is known in the art to be well tolerated and suitable for administration to human patients. Such salts include 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid (−L), malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulfuric acid, tartaric acid (+L), thiocyanic acid, toluenesulfonic acid (p), undecylenic acid. Pharmaceutically acceptable acids include hydrochloric acid and sulfuric acid.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “patient” is generally synonymous with the term “subject” and includes all mammals including humans. Preferably, the patient is a human.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

In certain embodiments, reference to “treating” or “treatment” of a subject at risk for developing a disease, or at risk of disease progression to a worse state, is intended to include prophylaxis. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “combination therapy” means the administration of two or more therapeutic agents to treat a therapeutic condition or disorder. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

Where a combination therapy is administered “simultaneously”, this includes treatment of a patient with a single dosage form (e.g. a tablet) comprising both liposomal annamycin and an additional anti-cancer substance; and also simultaneous dosing of separate dosage forms each separately comprising one of the respective combination partners.

Where a combination therapy is administered “sequentially” or “separately”, this includes treatment of a patient with a first dosage form liposomal annamycin followed by treatment of the same patient with a second dosage form comprising an additional anti-cancer substance; or treatment of a patient with a single dosage form comprising a particular anti-cancer substance, followed by treatment of the same patient with a second dosage form comprising liposomal annamycin. The interval between the sequential or separate doses may be judged by a skilled practitioner with reference to the information in this specification.

Further embodiments include the embodiments disclosed in the following Examples, which is not to be construed as limiting in any way.

### EXAMPLES

### Example 1: Determination of Pharmacokinetic and Biodistribution of L-Annamycin in Female CD-1 Mice

The pharmacokinetics and biodistribution of L-Annamycin was investigated in female CD-1 mice, after single bolus injection of L-Annamycin at 4 mg/kg. Animals were euthanized at several time points (n=5-8), followed by blood collection and extraction of following organs: lungs, kidneys, liver, hearts, brains, pancreas and spleens. Mouse tissues were homogenized at 100 mg/mL in methanol. Tissue homogenate was then extracted by protein precipitation using 10×volume of acetonitrile w/0.1% formic acid. The concentrations of annamycin in plasma and tissue lysates was performed using LC/MS/MS. Samples were analyzed using a Waters QuattroPremier XE mass spectrometer and Waters Acquity Classic UPLC. Detection was by electrospray negative ionization. Annamycin was chromatographically resolved using a Phenomenex Luna phenyl-hexyl 3 μm 2.1×150 mm column using a linear gradient of water and acetonitrile both containing 0.1% formic acid.

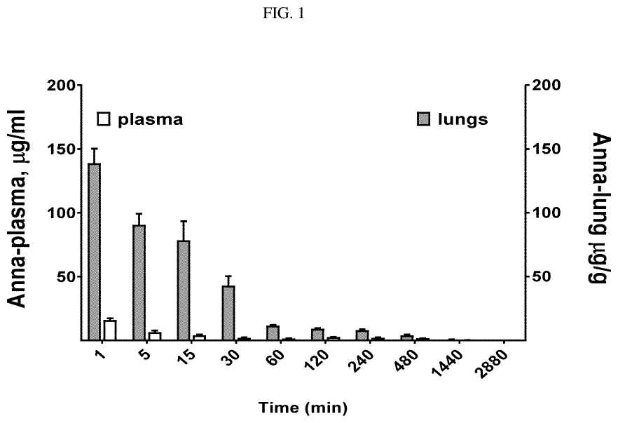

FIG. 1 presents the plasma and lungs levels of annamycin at different time points after L-Annamycin administration. The levels of the drug in lungs and plasma detected one minute after injection were 138.7 μg and 15.86 μg/ml, respectively, which corresponds to 216 uM and 25 uM, an unexpected finding. Significantly higher levels of annamycin in lungs than in plasma were detected throughout the entire study with the highest ratio of 28.7 recorded 30 min after drug administration. FIG. 1 shows PK and biodistribution analysis of annamycin after single intravenous administrations of the drug.

### Example 2: L-Annamycin Efficacy in Syngeneic “Lung Metastatic” CT26 Model of Colon Cancer

Efficacy of L-Annamycin (L-Ann) was tested in Balb/c injected intravenously with CT26 cells. Female Balb/c mice (8 weeks old) were injected iv with 2.5×10⁵ CT26WT-Luc-neo cells in 200 μl of PBS. Tumor progression was monitored using BLI. Bioluminescent signals were acquired 10 min after subcutaneous administration of D-Luciferin (100 μl of 15 mg/ml) using IVIS Lumina 100 Imager (FOV=25, F1, binning 8, automatic exposure time). The BLI parameters were kept unchanged during the entire study. The images were analyzed using Living Image software, version 4.5.5. For quantification, ROI was drawn over the entire mouse and normalize radiance unit (p/sec/cm²/sr) was used.

On day 17, the mice were randomized into three groups (n=11-12) receiving vehicle or L-Anna at 2 or 4 mg/kg (4 and 8 ml/kg respectively), given intravenously, once a week (5 doses total). The injected volume was adjusted to the body weight individually. Tumor growth was monitored using BLI imaging once a week. For continuous BLI analysis a max recorded BLI signal was assumed for dead mice. Distribution of the signals between the groups was analyzed using Kruskal-Wallis test.

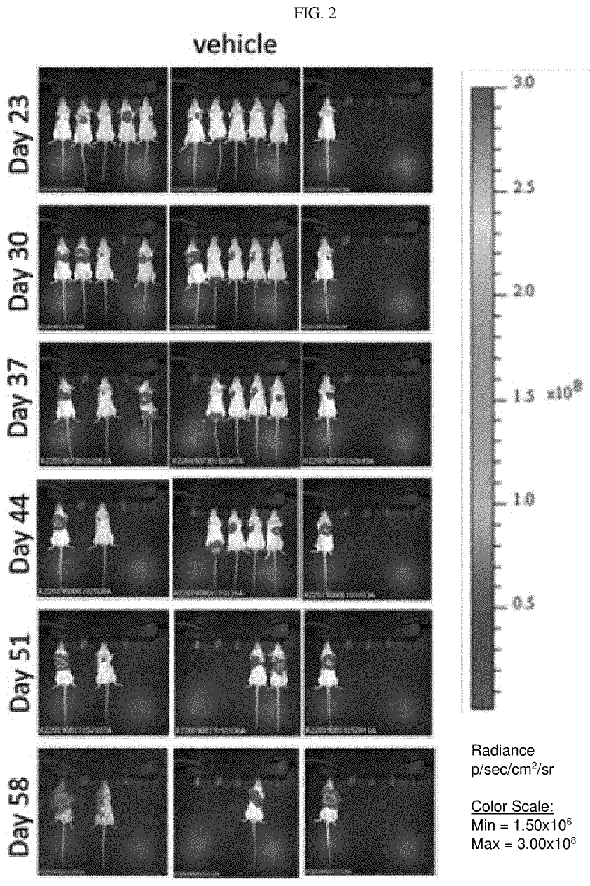

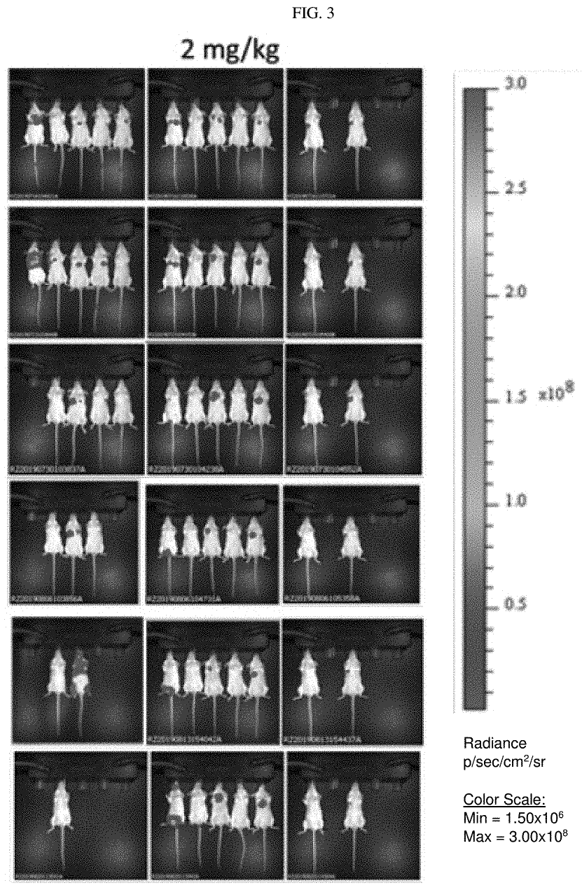

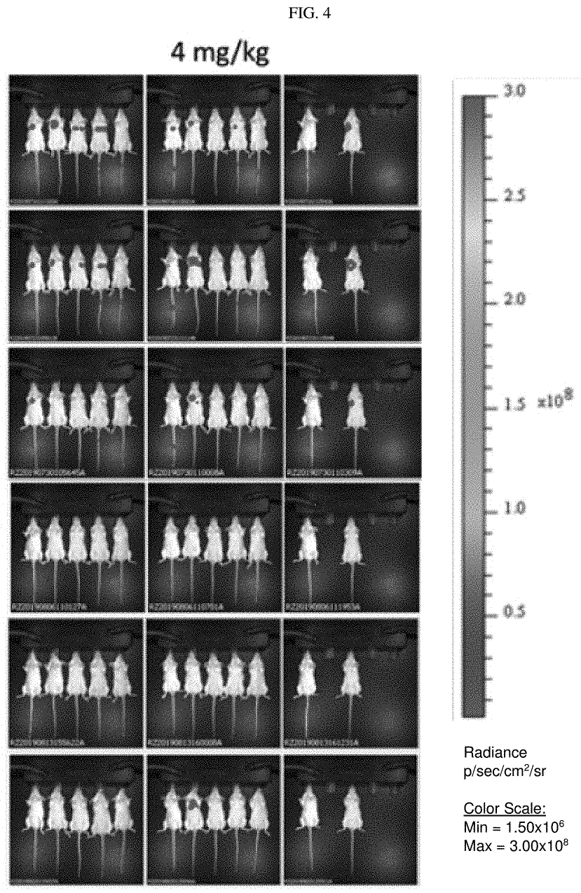

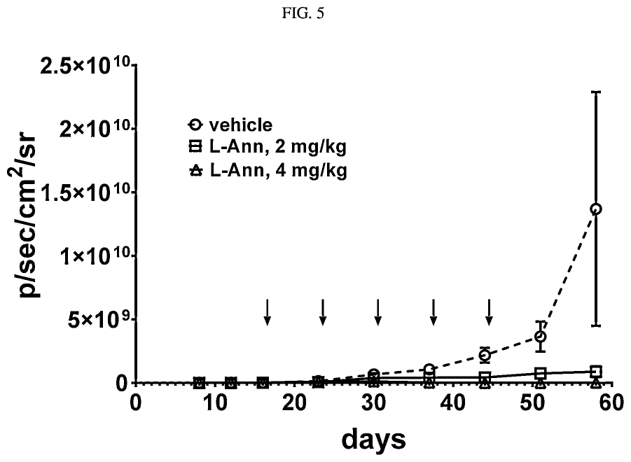

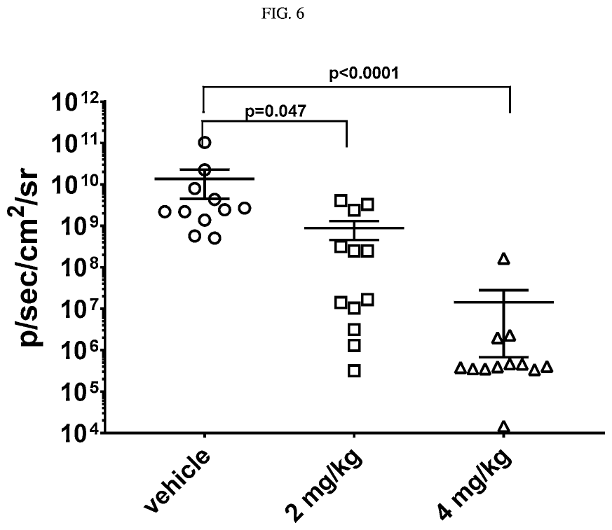

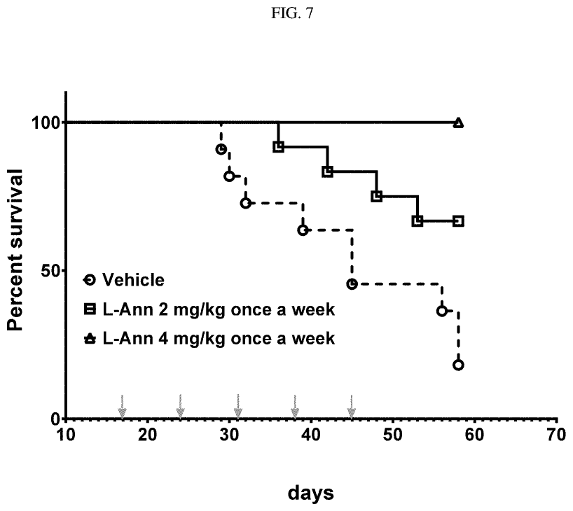

The tumor progression and survival of CT26 tumor-bearing mice treated with L-annamycin is shown in FIGS. 2-4. Based on bioluminescent imaging (BLI), the mice showed predominantly lung-localized tumors as shown in FIGS. 2-4. Dose-dependent delay in tumor progression in mice treated with L-Ann at 2 and 4 mg/kg was observed. Mouse on L-Anna 4 mg/kg schedule showed lasting tumor regression. FIGS. 2-4 and 5 represent a longitudinal analysis of BLI signal for mice treated with L-Ann at two different dosing levels: 2 and 4 mg/kg. FIG. 6 shows the distribution of BLI on day 58 after tumor implantation. Inhibition of tumor growth was clearly translated into improved survival of the mice: median survival of vehicle-treated mice was 46 days. Neither 2 or 4 mg/kg receiving groups reached mortality level allowing to establish median survival parameter yet; FIG. 7 shows percent survival by Kaplan-Mayer analysis of CT26 tumor-bearing mice treated with L-Annamycin. Of note, no deaths were reported in group treated with 4 mg/kg till day 58 of the study. In this experiment, median survival reached 117.5 days.

### Example 3: L-Annamycin Efficacy in Syngeneic “Lung Metastatic” 4T1 Model of Triple Negative Breast Cancer

The efficacy of L-Annamycin (L-Ann) was tested in Balb/c injected intravenously with 4T1 cells. Female Balb/c mice were injected iv with 2×10⁴ 4T1-Luc cells. Tumor growth was monitored using BLI imaging once a week. Bioluminescent signals were acquired 10 min after subcutaneous administration of D-Luciferin (100 μl of 15 mg/ml) using IVIS Lumina 100 Imager (FOV=25, F1, binning 8, automatic exposure time). The BLI parameters were kept unchanged during the entire study. The images were analyzed using Living Image software, version 4.5.5. For quantification, ROI was drawn over the entire mouse and normalize radiance unit (p/sec/cm²/sr) was used.

The treatment initiated 8 days post inoculation consisted of 6 doses of L-Ann at 4 mg/kg injected once a week. On day 8, the mice were randomized into two groups (n=10) receiving vehicle (saline 8 ml/kg) or L-Anna at 4 mg/kg (8 ml/kg). For continuous BLI analysis a max recorded BLI signal was assumed for dead mice. Survival curves were analyzed using Kaplan-Mayer analysis. P values were calculated using Log-rank (Mantel-Cox) test in Graph Pad Prism.

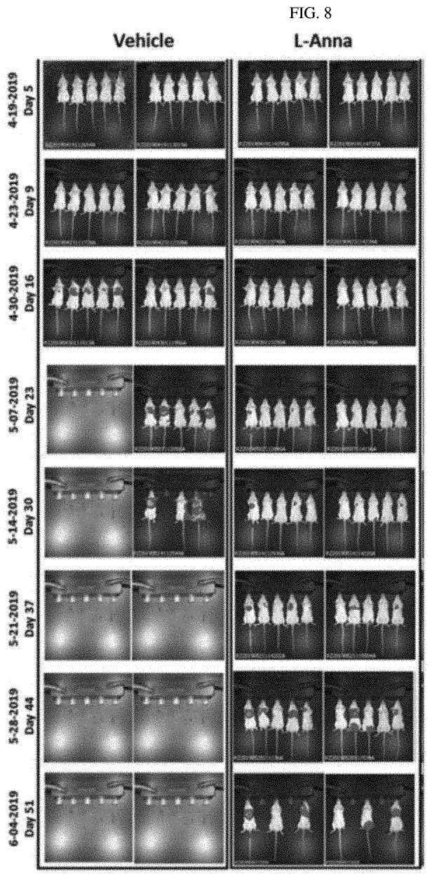

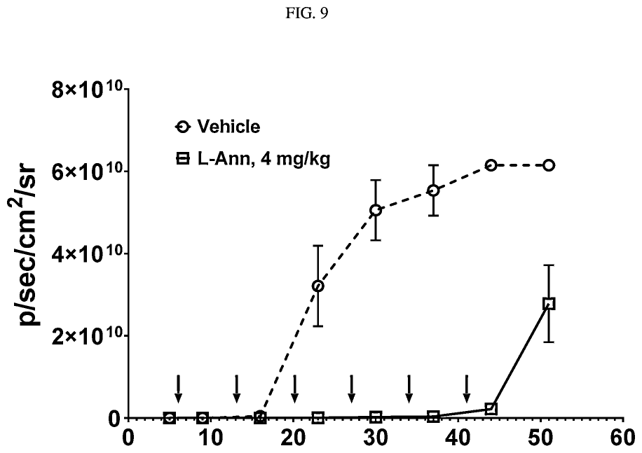

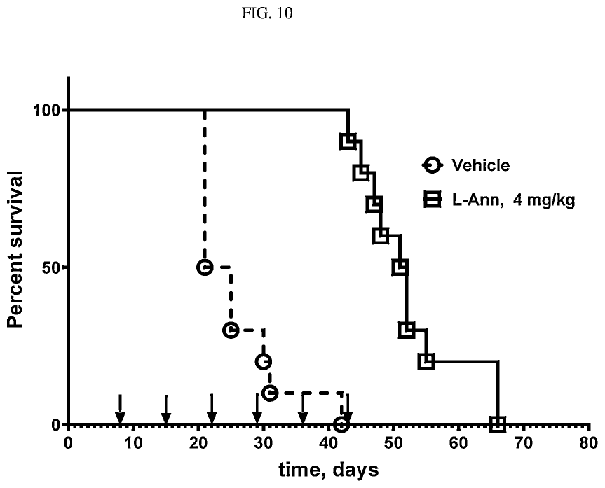

Based on bioluminescent imaging (BLI), the mice showed predominantly lung-localized tumors (FIG. 8). Clear delay in tumor progression in mice treated with L-Ann was observed. Treated group showed significantly lower bioluminescent signal when compared to animals receiving vehicle as early as 8 days after first dose administration (p=0.046) and continue to spread throughout the study (FIG. 8 and FIG. 9). Importantly, reduction of the tumor growth was correlated with improved survival: the median survival rate for the vehicle group was 23 days, while for L-Ann-treated mice it was extended to 53 days (survival ratio 2.26, p<0.0001, FIG. 10).

### Example 4: L-Annamycin Efficacy in MCA205 RFP Sarcoma Model

The efficacy of L-Annamycin (L-Ann) was tested in Balb/c injected intravenously with MCA205 cells. Female Balb/c mice were injected iv with 1×10⁵ MCA205 cells.

The treatment initiated 4 days post inoculation consisted of L-Ann at 4 mg/kg injected once a week. On day 4, the mice were randomized into two groups (n=10) receiving vehicle (saline 8 ml/kg) or L-Anna at 4 mg/kg (8 ml/kg). Survival curves were analyzed using Kaplan-Mayer analysis. P values were calculated using Log-rank (Mantel-Cox) test in Graph Pad Prism.

Clear delay in tumor progression in mice treated with L-Ann was observed. Importantly, reduction of the tumor growth was correlated with improved survival: the median survival rate for the vehicle group was 21 days, while for L-Ann-treated mice it was extended to 87.5 days (survival of 417% (% of control), p<0.0001).

### SUMMARY

As shown herein, annamycin accumulates rapidly in the lungs, and CD-1 mice receiving a single bolus injection of L-annamycin at 4 mg/kg achieved a high level of annamycin in lungs in just 1 minute after injection. Significantly higher levels of annamycin were detected in lungs than plasma through the entire study with the highest ratio of 28.7 recorded at 30 minutes after drug administration. See, Example 1 and FIG. 1. Not only does annamycin accumulate in the lungs, but it significantly reduces tumor growth and improves survival in three syngeneic “lung metastatic” mouse models of cancer as discussed further in Examples 2 (CT26 Colon Cancer), Example 3 (4T1 triple negative breast cancer), and Example 4 (MCA205 sarcoma).

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and methods.

1. A method of treating cancer in the lung comprising administering to a patient in need thereof a therapeutically effective amount of liposomal annamycin.

2. The method of claim 1, wherein said patient has primary or metastatic cancer in the lung.

3. The method of claim 2, wherein the primary lung cancer is non-small-cell lung carcinoma (NSCLC) or small-cell lung carcinoma (SCLC).

4. The method of claim 3, wherein the primary lung cancer is a non-small-cell lung cancer selected from adenocarcinoma, squamous-cell carcinoma, and large-cell carcinoma.

5. The method of claim 2, wherein said patient has metastatic cancer in the lung.

6. The method of claim 5, wherein the metastatic cancer is a metastasis of a primary cancer selected from bladder cancer, breast cancer, colorectal cancer, head and neck cancer, kidney cancer, melanoma, pancreatic cancer, prostate cancer, and ovarian cancer.

7. The method of claim 5, wherein the metastatic cancer is a metastasis of a sarcoma.

8. The method of claim 5, wherein the metastatic cancer is from a cancer of unknown primary origin.

9. The method of claim 1, further comprising administering an effective amount of at least one chemotherapeutic agent to the subject.

10. The method of claim 9, wherein the at least one chemotherapeutic agent is selected from actinomycin, afatinib, alectinib, asparaginase, azacitidine, azathioprine, bicalutamide, bleomycin, bortezomib, camptothecin, carboplatin, capecitabine, certinib, cetuximab,cisplatin, chlorambucil, crizotinib, cyclophosphamide, cytarabine, daunorubicin, docetaxel, doxifluridine, doxorubicin, erlotinib, epirubicin, epothilone, etoposide, fludarabine, flutamine, fluorouracil, fostamatinib, gefitinib, gemcitabine, hydroxyurea, ibrutinib, idarubicin, ifosfamide, imatinib, ipilimumab, irinotecan, lapatinib, letrozole, mechlorethamine, mercaptopurine, methotrexate, mitomycin, mitoxantrone, nilotinib, octreotide, oxaliplatin, paclitaxel, palbociclib, panitumumab, pemetrexed, raltitrexed, selumetinib, sorafenib, sunitinib, tamoxifen, temozolomide, teniposide, tioguanine, topotecan, trastuzumab, tremelimumab, valrubicin, vemurafenib, vinblastine, vincristine, vindesine, vinorelbine, and combinations thereof.

11. The method of claim 9, wherein the at least one chemotherapeutic agent comprises a combination selected from:
cyclophosphamide, doxorubicin, and vincristine;
mitomycin, vindesine and cisplatin;
cisplatin and vinorelbine; and
cisplatin, etoposide and ifosfamide.

12. The method of claim 1, further comprising administering an effective amount of at least one immunotherapeutic agent to the subject.

13. The method of claim 1, further comprising one or both of resecting the lung cancer and administering radiation therapy.

14. The method of claim 1, wherein treating cancer in the lung comprises one or more of the following: (a) increasing survival time of the patient; (b) reducing volume of the primary cancer; (c) retarding growth of the primary cancer; (d) reducing number of metastatic tumors; (e) reducing volume of metastatic tumors; and (f) retarding growth of metastatic tumors.

15. The method of claim 1, wherein treating cancer in the lung does not result in annamycin-induced toxicity of the lung of such severity that repeated administration of the annamycin is contraindicated.

16. The method of claim 1, wherein treating cancer in the lung does not result in annamycin-induced systemic toxicity of such severity that repeated administration of the annamycin is contraindicated.

17. The method of claim 1, wherein said administering is repeated weekly.

18. The method of claim 1, wherein said administering is repeated every two, three, or four weeks.

19. The method of claim 1, wherein the liposomal annamycin comprises annamycin, one or more lipids, and one or more non-ionic surfactants.

20. The method of claim 19, wherein the lipids comprise Dimyristoylphosphatidylcholine (DMPC) and dimyristoylphosphatidylglycerol (DMPG).

21. The method of claim 19, wherein the non-ionic surfactant comprises a polysorbate-type surfactant formed from the ethoxylation of sorbitan followed by the addition of a carboxylic acid.

22. The method of claim 21, wherein the non-ionic surfactant comprises polyoxyethylene sorbitan monolaurate.

23. The method of claim 1, wherein the liposomal annamycin is provided as a preliposomal lyophilizate composition that is reconstituted into an aqueous liposome composition through hydration prior to administration.

24. The method of claim 23, wherein the preliposomal annamycin lyophilizate comprises:
1.8-2.2 wt % annamycin;
3.0-3.4 wt. % Polysorbate 20; and
94.4-95.2 wt. % of lipids selected from DMPC and DMPG.

25. The method of claim 24, wherein the DMPC is 65.3-67.3 wt. % and the DMPG is 27.1-29.9 wt. %.
